(19) United States
(12) Patent Application Publication
(10) Pub. No.: US 2011/0230878 A1
(43) Pub. Date: Sep. 22, 2011

(54) Ablation Handle Attachment

(76) Inventors: Shawn Ryan, Upton, MA (US); Caroline Gaudet, West Roxbury, MA (US); Andrew Whitney, Douglas, MA (US)

(21) Appl. No.: 13/038,743

(22) Filed: Mar. 2, 2011

Related U.S. Application Data
(60) Provisional application No. 61/314,422, filed on Mar. 16, 2010.

(51) Int. Cl. A61B 18/12

(52) U.S. Cl. 606/41

(57) ABSTRACT

A device for use in conjunction with a medical device comprises a handle extending longitudinally from a proximal end to a distal end and including a lumen extending therethrough for accommodating a longitudinal probe. A distal end of the handle including a coupling for attachment to a proximal end of a needle device. The handle includes a length adjusting mechanism enabling a user to set a length of the handle at a desired length and a flexible probe sized and shaped for insertion through a lumen of the medical device. A length of the probe is selected relative to a length of the lumen within which it is to be employed so that, when the length of the handle is set at a desired length, insertion of the flexible probe through the lumen to a distal-most position extends a distal portion of the probe distally beyond the distal end of the lumen by a desired distance.

### PRIORITY CLAIM

The application claims the priority to the U.S. Provisional Application Ser. No. 61/314,422, entitled “ABLATION HANDLE ATTACHMENT” filed Mar. 16, 2010. The specification of the above-identified application is incorporated herewith by reference.

### BACKGROUND

be desirable to perform one or more therapeutic procedures in conjunction with an endoscopic fine needle aspiration procedure. Users may wish to perform any of including, but not limited to, ablation, lithotripsy, photodynamic therapy, coagulation, vaporization, incision or a combination of any of these procedures in a region from which tissue is being aspirated. For example, these procedures may be performed utilizing technologies including, but not limited to, electrohydraulic lithotripsy (EHL) or any of a variety of ablation technologies including laser, cryotherapy, argon plasma or radio frequency ablation, and argon plasma. However, minimally invasive applications of these therapies have been limited because of difficulty navigating tortuous anatomy and the inability to easily visualize distal tips of the therapeutic probes. Ablation devices have been employed which access target areas along tortuous paths for example via insertion through flexible EUS FNA devices. However, the addition of such devices to the apparatus for EUS FNA device and the endoscope through which the EUS FNA device is inserted into the body increases the complexity and difficulty of these procedures.

### SUMMARY OF THE INVENTION

The present invention is directed to a device for use in conjunction with a medical device, comprising a handle extending longitudinally from a proximal end to a distal end and including a lumen extending therethrough for accommodating a longitudinal probe, a distal end of the handle including a coupling for attachment to a proximal end of a needle device such as a fine needle aspiration device, the handle including a length adjusting mechanism enabling a user to set a length of the handle at a desired length and a flexible probe sized and shaped for insertion through a lumen of the medical device, a length of the probe being selected relative to a length of the lumen within which it is to be employed so that, when the length of the handle is set at a desired length, insertion of the flexible probe through the lumen to a distal-most position extends a distal portion of the probe distally beyond the distal end of the lumen by a desired distance.

### BRIEF DESCRIPTION OF THE DRAWINGS

FIG. 1 shows a perspective view of a device according to an exemplary embodiment of the present invention;

FIG. 2 shows a side plan view of the device of FIG. 1; and

FIG. 3 shows a cross-sectional side view of the device of FIG. 1.

### DETAILED DESCRIPTION

The present invention may be further understood with reference to the following description and the appended drawings, wherein like elements are referred to with the same reference numerals. The present invention relates to an attachment mechanism for coupling a therapeutic device to an endoscopic device. In particular, exemplary embodiments of the present invention describe ablation probe handle attachments configured for attachment to a medical device to improve visibility of distal ends of ablation devices while eliminating the burden of manipulating a therapeutic device in addition to the medical device. Although exemplary embodiments are specifically described herein including the attachment of an ablation probe, it will be understood by those of skill in the art that the attached device may be any therapeutic device. It will be understood by those of skill in the art that the present invention may relate to any therapeutic device that may be inserted through, for example, an endoscope, any of a variety of different types of catheters, etc. It should be noted that the teens “proximal” and “distal” as used herein, are intended to reference a direction toward (proximal) and away from (distal) a user of the device.

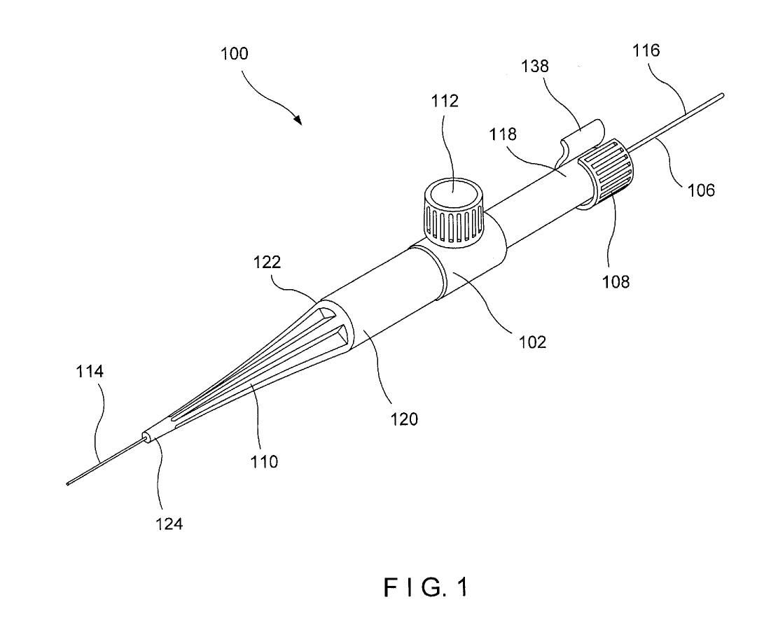

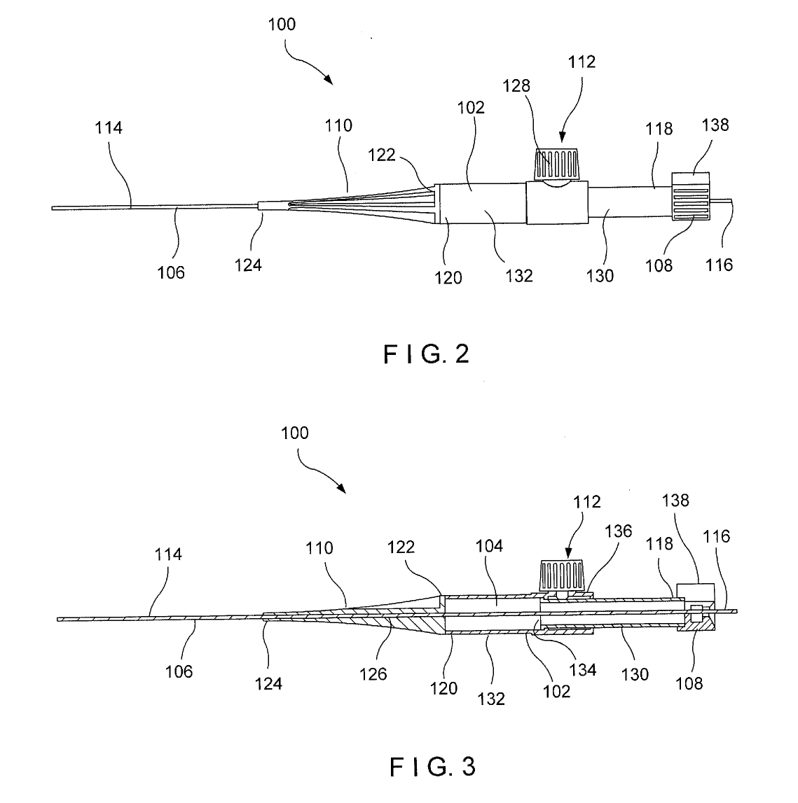

As shown in FIGS. 1-3, an ablation device 100 according to an exemplary embodiment of the invention comprises a handle 102 including a lumen 104 extending therethrough and an attachment mechanism 108 for coupling the ablation device 100 to a medical device (not shown) including but not limited to a needle, an EUS FNA device, an ERCP catheter, a stent delivery catheter, an endoscope, a balloon catheter, a dilator, an introducer, a guide sheath, etc. The ablation device 100 may further comprise a strain relief 110 preventing damage to a proximal portion 114 of a probe 106 extending proximally from the handle 102 and a length adjusting mechanism 112 to control a length of a distal portion 116 of the probe 106 extending distally from the handle 102. The probe 106 may be an electrode or, alternatively, may include an electrode that extends from a distal end of the probe 106 to ablate a target area. Alternatively, the probe 106 may include an electrode formed of a conductor that is ‘painted’ on a surface of the probe 106. The electrode may be, for example, a needle extending from the probe 106 or an array of electrode tines extending distally from the probe 106. It will be understood by those of skill in the art, however, that the electrode may take a variety of forms so long as the electrode is capable of ablating the target area and extends from a distal end of the probe 106.

The handle 102 extends longitudinally from a distal end 118 to a proximal end 120, the attachment mechanism 108 connected to the distal end 118. The probe 106 extends through the lumen 104 of the handle 102 with the distal portion 116 extending distally from the distal end 118 for inserted through a channel of a medical device, while the proximal portion 114 extends proximally from the proximal end 120 to be connected to a source of energy (e.g. an RF generator (not shown)) to provide an ablative energy to the distal end of the probe 106 as would be understood by those skilled in the art.

The distal portion 116 is inserted through the channel of the medical device until the attachment mechanism 108 comes into contact with and couples to a proximal end (e.g., a port) of the medical device. The attachment mechanism 108 may be a luer such as a male swivel for receiving a proximal end of the medical device or a snap type attachment snapping onto a portion of the proximal end thereof. However, it will be understood by those of skill in the art that the attachment mechanism 108 may take any of a variety of forms so long as the attachment mechanism 108 is able to couple the ablation device 100 to the proximal end of the medical device with the lumen 104 of the handle 102 in alignment with the channel of the medical device. In a further embodiment, the attachment mechanism 108 may, for example, be a Y-connector facilitating fluid removal and/or injection through the medical device. It will also be understood by those of skill in the art that attaching the ablation device 100 to the medical device facilitates the user's manipulation of the diagnostic and therapeutic devices—i.e., because the ablation device 100 is coupled to the medical device, the user can move both of these devices together without the need to grasp them separately.

The distal portion 116 is inserted into the channel of the medical device until a distal end of the probe 106 is in a desired position relative to a distal end of the medical device, enabling the user to position the distal end 116 of the ablation device 100 accurately by observing the distal end of the medical device under ultrasound. Specifically, the distal end 116 of the probe 106 includes features such as, for example, markings, to improve ultrasound visibility thereof by enhancing the echogenic signal reflected therefrom. It will be understood by those of skill in the art that the distal portion 116 is formed of a material having sufficient flexibility to enable its insertion through the channel of a medical device which has itself been inserted along a tortuous path to the target area.

The ablation device 100 includes a length adjust mechanism 112 permitting the user to adjust a length of the distal portion 116 such that the position of the distal end thereof relative to a distal end of the medical device may be adjusted as desired. That is, the length of the distal portion 116 may be adjusted so that a length of the distal portion 116 which projects distally beyond a distal end of the medical device when fully extended is a desired length. For example, the length adjust mechanism 112 may include a knob 128, which may be rotated to loosen and/or tighten a coupling between a first portion 130 and a second portion 132 of the handle 102 such that the first and second portions 130, 132 may be moved relative to one another in a telescoping assembly. The first portion 130 may be co-axially coupled to the second portion 132 such that a proximal end 134 of the first portion 130 is received within a distal end 136 of the second portion 132. Rotating the knob 128 in a first direction loosens the coupling such that the first portion 130 may be slid longitudinally into and out of the second portion 132, adjusting the length of the distal portion 116 extending distally from the distal end of the medical device. Once the length of the distal portion 116 has been adjusted as desired, the knob 128 may be rotated in a second direction to fix the first and second portions 130, 132 relative to one another, thereby fixing the length of the distal portion 116. The knob 128 may also be associated with an indicator that shows a depth of penetration of the first portion 130 within the second portion 132. It will be understood by those of skill in the art that other relative movement of the first and second portions 130, 132 is also be possible. For example, the first portion 130 may be moved longitudinally into the distal end 136 of the second portion 132 via a rotation relative to the second portion 132 such that a threading along an outer surface of the first portion 130 engages with a threading along an inner surface of the second portion 132. Alternatively, the first and second portions 130, 132 may be secured to one another via a locking lever such as, for example, a ratchet. The handle 102 may also include graduated markings along a length thereof indicating changes in length of the distal portion 116 such that the user can determine the extent of movement of the length adjust mechanism 112 necessary to achieve a desired position of the distal end of the probe 106 relative to the distal end of the medical device.

The handle 102 provides adequate support of the probe 106 to prevent buckling of the probe 106. Specifically, the probe 106 is constrained within the telescoping assembly of the handle 102. By constraining an outer diameter of the probe 106, the telescoping assembly facilitates advancement of the probe 106 through the handle while preventing buckling thereof. The telescoping assembly (e.g., the first and second portions 130, 132) may be constructed of metal components, integrated into injection molding components or various other methods.

The handle 102 may also include a clip 138 at the distal end 118 for clipping a working length of the distal portion 116 during handling of the probe 106. The distal portion 116 may, for example, be clipped in a coiled configuration such that the probe 106 is maintained in a compact configuration as the distal portion 116 is inserted into the body. Thus, coils of the distal portion 116 may be released from the clip 138 as the distal portion 116 is inserted farther into the body to the target area. Additionally, the clip 138 may be used to clip the distal portion 116 as the distal portion 116 is withdrawn from the body. Thus, the clip 138 permits convenient storage of the probe 106. The clip 138 may be formed, for example, as described in U.S. Provisional Patent Application Ser. No. 61/121,815 to Ryan et al., entitled “Clip for Handling an Endoscopic Device” the entire disclosure of which is hereby expressly incorporated by reference herein.

A strain relief 110 which is connected to the proximal end 120 of the handle 102 may taper from a maximum diameter at a distal end 122 to a minimum diameter at a proximal end 124 and may include a lumen 126 extending therethrough sized and shaped to slidably accommodate the proximal portion 114 of the probe 106 extending proximally from the proximal end 122 of the handle 102. An outer diameter of the tapered proximal end 124 is only slightly larger than a diameter of the probe 106 such that the strain relief 110 provides support to the proximal portion 114 as the proximal portion 114 extends from the rigid handle 102 to an unconstrained environment. The strain relief 110 is formed of a substantially flexible material to permit movement of the proximal portion 114 relative to a longitudinal axis of the handle 102. In a further embodiment, the device 100 may further include a sheath (not shown) slidable over the proximal portion 114 of the probe 106 to provide additional support and increase durability of the unsupported proximal portion 114.

In an alternate embodiment, the strain relief 110 is removably coupled to the proximal end 120 of the handle 102 such that the probe 106 may be inserted through the lumen 104 of the handle 102 after the handle 102 has been attached to the medical device via the attachment mechanism 108. The strain relief 110 may be coupled to the handle 102 via for example a friction or snap fit. An insertion feature including an adjustable diameter silicone gasket may be attached to the proximal end 120 such that the probe 106 may be held in a desired position within the handle 102 once the probe 106 has been inserted therethrough. The strain relief 110 may also include a slit along a longitudinal axis thereof for receiving the probe 106 therethrough while maintaining a friction fit with the probe 106 to control a position of the probe 106 relative to the strain relief 110 once assembled.

The device 100, as described above, may be used to treat, for example, biliary duct tumors, lung tumors, liver tumors and GI tract tumors. In some cases, the tumor may be in the same duct or area as the device 100. In other cases, the tumor may be reachable by piercing through an organ—e.g., through the GI tract to the liver or to a tumor outside the tract—via a needle of the medical device to which the probe 106 is attached. It will also be understood by those of skill in the art that although the exemplary embodiments of the present invention specifically describe a device attaching an ablation probe to a medical device such as, for example, a needle device, the present invention is directed to an attachment mechanism that facilitates attachment between any therapeutic device and any medical device. For example, the present invention may also facilitate attachment between an EHL probe and a catheter such as, for example, the SpyScope® Access and Delivery Catheter (Boston Scientific Corporation).

It will be apparent to those of skill in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A device for use in conjunction with a medical device, comprising:
a handle extending longitudinally from a proximal end to a distal end and including a lumen extending therethrough for accommodating a longitudinal probe, a distal end of the handle including a coupling for attachment to a proximal end of a needle device, the handle including a length adjusting mechanism enabling a user to set a length of the handle at a desired length; and
a flexible probe sized and shaped for insertion through a lumen of the medical device, a length of the probe being selected relative to a length of the lumen within which it is to be employed so that, when the length of the handle is set at a desired length, insertion of the flexible probe through the lumen to a distal-most position extends a distal portion of the probe distally beyond the distal end of the lumen by a desired distance.

2. The device of claim 1, wherein the probe comprises an electrode.

3. The device of claim 1, further comprising:
a strain relief connected to the proximal end of the handle.

4. The device of claim 3, wherein the strain relief tapers from a maximum diameter at a distal end thereof to a minimum diameter at a proximal end and including a lumen extending therethrough sized and shaped to slidably receive a portion of the flexible probe extending proximally from the handle.

5. The device of claim 1, wherein the handle includes a first member telescopically received within a second member so that, as the first member is moved relative the second member a length of the handle is adjusted.

6. The device of claim 5, wherein the length adjusting member includes a locking mechanism operable by a user to lock the positions of the first and second members relative to one another in a desired position.

7. The device of claim 5, wherein the first member includes threads about an outer surface thereof mating with threads along an inner surface of the second member so that rotation of the first member relative to the second member moves the first member axially within the second member to be received therein.

8. The device of claim 5, wherein the handle further includes markings along a length thereof indicating, for the current length of the handle, a length by which the probe will extend distally from the distal end of the lumen.

9. The device of claim 1, wherein the coupling includes a luer adapted to receiving a proximal end of a medical device.

10. The device of claim 1, further comprising:
a clip on the handle configured to clip a working length of the probe thereto to maintain the probe in a compact configuration before it is utilized.

11. The device of claim 1, wherein the end effector delivers one of RF energy, laser energy, cryogenic energy and argon plasma energy.

12. The device of claim 10, wherein the end effector comprises an ablation element and the probe includes an electrically conductive element transmitting ablation energy from a source coupled to the proximal end of the probe to the ablation element.

13. An attachment system for coupling a device to a medical device, comprising:
a handle extending longitudinally from a proximal end to a distal end and including a lumen extending therethrough, the handle including a coupling at the distal end adapted to couple the handle to a proximal end of a handle of a medical device and a length adjustment mechanism adjusting a length of the handle to a desired length: and
a flexible probe including a therapeutic device at a distal end thereof, the probe being sized and shaped to be slidably inserted through the lumen of the handle and into a lumen of a needle of the medical device.

14. The system of claim 13, further comprising:
a strain relief connected to the proximal end of the handle.

15. The system of claim 14, wherein the strain relief tapers from a maximum diameter at a distal end thereof to a minimum diameter at a proximal end and including a lumen extending therethrough sized and shaped to slidably receive a portion of the flexible probe extending proximally from the handle

16. The system of claim 13, wherein a distal end of the probe includes an echogenic surface to improve a visibility of the probe under ultrasound imaging.

17. The system of claim 13, wherein the handle further comprises a clip for clipping a portion of the probe extending proximally from the handle to maintain the probe in a compact configuration.

18. A device for attaching an ablation probe to a needle device, comprising:
a handle extending longitudinally from a proximal end to a distal end and including a lumen extending therethrough for accommodating the ablation probe slidably therethrough, the handle including a coupling for attaching the distal end of the handle to a proximal end of the needle device; and
a flexible probe including a therapeutic device at a distal end thereof, the probe being sized and shaped to be slidably inserted through the lumen of the handle and into a lumen of the needle device.

19. The device of claim 18, further comprising:
a strain relief connected to the proximal end of the handle.

20. The device of claim 19, wherein the strain relief tapers from a maximum diameter at a distal end thereof to a minimum diameter at a proximal end and including a lumen extending therethrough sized and shaped to slidably receive a portion of the flexible probe extending proximally from the handle.

21. The device of claim 18, wherein the handle includes a length adjustment mechanism enabling a user to adjust a length of the handle to a desired length relative to a length of the flexible probe so that, when the probe is inserted through the needle device to a distal-most position, the therapeutic device projects distally beyond a distal end of the needle by a desired distance.

22. The device of claim 18, further comprising:
a clip at the distal end of the handle configured to clip a portion of the probe extending proximally from the handle to maintain the probe in a compact configuration until it is inserted into the fine needle aspiration device.

23. The device of claim 18, wherein the probe comprises an RF electrode.

24. The device of claim 18, wherein the probe comprises a laser fiber.
